(19) United States
(12) Patent Application Publication
(10) Pub. No.: US 2026/0166511 A1
(43) Pub. Date: Jun. 18, 2026

(54) Hollow silicone resin particles and method for the production thereof

(71) Applicant: Wacker Chemie AG, Munich (DE)

(72) Inventor: Sebastian KNÖR, München (DE)

(73) Assignee: Wacker Chemie AG, Munich (DE)

(21) Appl. No.: 19/124,300

(22) PCT Filed: Dec. 19, 2022
(86) PCT No.: PCT/EP2022/086714

§ 371 Date: Apr. 25, 2025

(51) Int. Cl. B01J 13/18; A61K 8/02; A61K 8/891; A61Q 19/00; C08G 77/00; C08G 77/06; C08G 77/16; C08K 9/06; C08L 83/04; C09D 7/40; C09D 7/62; C09D 7/65; C09D 183/04

(52) U.S. Cl. CPC B01J 13/185; A61K 8/0279; A61K 8/891; A61Q 19/008; C08G 77/06; C08G 77/16; C08K 9/06; C08L 83/04; C09D 7/62; C09D 7/65; C09D 7/68; C09D 7/69; C09D 7/70; C09D 183/04; C08G 77/80; C08K 2201/006; C08L 2205/025; C08L 2205/20

(57) ABSTRACT

Hollow particles P and processes for producing the same. Where the hollow particle P is constructed of a hollow core K and a shell H which is made of a silicone resin composition Z that includes a condensation-crosslinked silicone composition X and a particulate solid F.

The invention relates to hollow particles constructed of a hollow core and a shell of a condensation-crosslinked silicone resin and particulate solid, and a to a process for the production thereof.

Hollow-body particles are widely used for example as lightweight fillers for reducing the density of polymer or ceramic components, or for absorbing, transporting, and releasing active constituents, such as fragrances, care substances or active substances, for example in cosmetic or medical applications or in household products or detergents.

The production of hollow-body particles is very laborious and usually uses a hard or soft template, on which the shell is constructed and which is then removed again very laboriously. These methodologies are ecologically and economically extremely disadvantageous.

WO2007113095 and WO2021121562 describe polysiloxane-based core-shell particles that have very advantageous properties in cosmetic applications and as an additive in numerous technical applications, where they have advantages due to surface effects in particular. However, the filled structure means that these particles are unable to absorb functional substances in their interior.

US2009004418 describes hollow silicone resin particles having a particle size of less than 1 mm, in which the shell is formed of a silicone resin composition of SiO_{4/2} units, RSiO_{3/2} units, and R₂SiO_{2/2} units. Production is very laborious and ecologically and economically extremely disadvantageous, since either a template particle, for example an organic polymer particle, and/or a toxic organic solvent, for example toluene or xylene, is initially suspended in water and then coated with reactive silanes to form the silicone resin shell, the core being removed with an organic solvent in a final step.

U.S. Pat. No. 9,802,175 B2 describes a process for producing hollow silicone resin particles constructed of RSiO_{3/2} units and having a particle size less than 200 nm. Production is very laborious and ecologically and economically extremely disadvantageous, since an organic template particle, for example a polystyrene particle, a polyacrylate particle or a polyvinyl acetate particle, is initially produced, which in a second step is coated with alkoxy-functional silanes to form the silicone resin shell, the core being removed with an organic solvent and heat in a third step.

U.S. Pat. No. 5,945,043 describes a process for producing hollow polysiloxane particles having a shell formed from a thermoplastic polysiloxane. The thermoplastic polysiloxane is dissolved in a solvent and the mixture is dispersed in water. The dispersion is sprayed with a spray dryer, resulting in removal of solvent and water and formation of the hollow thermoplastic polysiloxane particles. The polymer shell of the particles is not crosslinked. As a result, such particles are sensitive to temperature and solvents. The use of a toxic solvent makes production ecologically and economically extremely disadvantageous.

WO14098107 describes a process in which silica particles are used as a template for producing hollow polysiloxane particles. The silica particles undergo dispersion. On the surface of the template particles, the polysiloxane shell is formed through hydrolysis and condensation of an alkoxy-functional silane or siloxane. The template particle is then detached and broken down. The process is ecologically and economically extremely disadvantageous, since the silica particles used as templates and which are detached and broken down in the final step of the process are very laborious to produce.

Xue Wang et al. (Journal of Colloid and Interface Science 542 (2019) 144-150) describe a process for producing hollow particles via a Pickering emulsion. In this process, a solution of a photopolymerizable compound in an organic oil is emulsified in an aqueous phase, the boundary phase being stabilized by finely particulate silica particles. In a second process step, the photopolymerizable compound is polymerized at the boundary phase and forms a solid shell together with the silica particles. The use of an organic oil as template makes production ecologically and economically extremely disadvantageous.

All the abovementioned processes have the disadvantage of employing a solid or liquid template compound to construct a core-shell structure that is coated and then has to be laboriously removed and disposed of.

The invention provides hollow particles P constructed of a hollow core K and a shell H comprising a silicone resin composition Z that comprises condensation-crosslinked silicone composition X and particulate solid F.

The median particle diameter d50 of the hollow particles P is in the range 0.1-100 μm, preferably in the range 0.4-60 μm, and preferably in the range 0.8-40 μm.

The hollow particles P are preferably essentially spherical. The sphericity SPHT3 is preferably at least 0.8, more preferably at least 0.82, determinable in accordance with ISO 9276-6 using a Camsizer X2 from Retsch Technology.

The hollow particles P are amphiphilic, have a defined and uniform structure, and can be dispersed in aqueous and oily media.

The hollow particles P have the further advantage that their low density as an additive in formulations means they migrate to the surface and accordingly display enhanced surface effects. They are also suitable for use as a lightweight filler, for example for ceramics.

The hollow particles P can hold other substances. Filled particles are unable to do this.

The invention also provides a simple and inexpensive process for producing the hollow-body particle that does not involve the use of a template.

The invention further provides a process for producing the hollow particles P in which, in a first step, a dispersion V comprising particulate solid F and water is mixed with condensation-crosslinkable silicone composition X1, which comprises alkoxy-group-containing silicone resin A that is liquid at 20° C. and alkoxy-group-containing silane B, to form a continuous water-containing phase and a discontinuous phase comprising condensation-crosslinkable silicone composition X1,
and, in a second step, the silicone composition X1 undergoes crosslinking in the discontinuous phase to form the silicone composition X, resulting in the formation of the hollow particles P.

The advantageous process of the invention differs from prior art processes particularly in that it does not involve the use of a liquid or solid template. The templates used according to the prior art form a core on the surface of which a shell is constructed. The template is then removed again, with the formation of a hollow particle. According to the process of the invention, the shell H is formed through condensation-crosslinking of the emulsified condensation-crosslinkable silicone composition X1 at the boundary phase to the water-containing continuous phase of the emulsion E. The emulsified droplets of the condensation-crosslinkable silicone composition X1 thus initially form a temporary core, on the surface of which the condensation-crosslinkable silicone composition X1 then combines with particulate solid F to form a shell H during crosslinking to silicone composition X, with the formation of the hollow particle P. With the process of the invention, there is no need for any separate, costly or laborious to produce template that must be laboriously separated, recycled or disposed of as waste. The process of the invention is accordingly economically and ecologically very advantageous.

The condensation-crosslinkable silicone composition X1 preferably comprises, respectively based on the total amount of components (A) and (B),
(A) 50-90% by weight of at least one silicone resin A,
   composed of units of formulas (Ia), (Ib), (VII), and (Id)

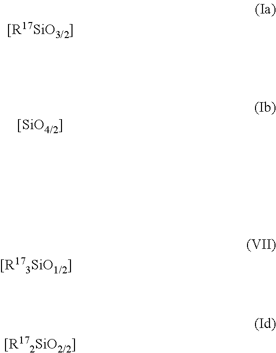

where
R¹⁷ represent identical or independently different monovalent, substituted or unsubstituted organic radicals bearing or not bearing functional groups, or an —OH or a hydrogen radical,
   with the provisos that
at least 20 mol % of the formula (Ia) or (Ib) or of a mixture of the two is present in (A),
not more than 50 mol % of the formula (Ib) is present in (A),
alkoxy groups are present as R¹⁷ in (A) to an extent of at least 5% by weight, with the proviso that
(A) is liquid at 20° C.,
(B) 10-50% by weight of at least one silane B of the general formula

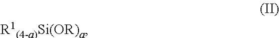

in which
R is a hydrocarbon radical having 1 to 16 carbon atoms, the carbon chain of which may be interrupted by non-adjacent —O— groups,
R¹ represents monovalent hydrocarbon radicals that are identical or independently different from one another, and
a represents the values 2, 3 or 4, where
   at least 20% by weight of silanes B, based on the total mass of all silanes
   B, satisfy the characteristic a=3 or 4.

### Component (A)

The condensation-crosslinkable silicone composition X1 used according to the invention preferably contains 55-85% by weight of one or more silicone resins A, preferably 60% to 80% by weight, in each case based on the total amount of components (A) and (B).

The silicone resins A are preferably ones having a molecular weight Mw of at least 500, preferably at least 600, more preferably at least 700, and not more than 5000, preferably not more than 4000, more preferably not more than 3000, wherein the polydispersity is not more than 20, preferably not more than 18, more preferably not more than 16, in particular not more than 15.

The silicone resins A contain at least 20 mol %, preferably at least 30 mol %, more preferably at least 40 mol %, in particular at least 50 mol %, of repeat units of the formula (Ia) or (Ib) or of a mixture of formulas (Ia) and (Ib), wherein repeat units of the formula (Ib) are present in an amount of not more than 50 mol %, preferably not more than 40 mol %, more preferably not more than 20 mol %. In a particularly preferred embodiment, no units (Ib) are present in the silicone resins A.

Repeat units of the formula (Id) may be present in the silicone resins A in an amount of up to 80 mol %, preferably up to 70 mol %, more preferably up to 60 mol %, in particular up to 50 mol %.

The silicone resins A contain alkoxy groups as R 17 to an extent of at least 5% by weight, preferably at least 8% by weight, and particularly preferably at least 10% by weight.

Examples of suitable alkoxy groups as R¹⁷ are hydrocarbonoxy radicals having 1 to 16 carbon atoms, which can also be substituted. Particularly suitable and thus preferred are methoxy, ethoxy, isopropoxy, n-butoxy, and tert-butoxy radicals and the p-nitrophenoxy radical, with particular preference given to the methoxy and ethoxy radical.

All other R 17 may independently of one another be monovalent, substituted or unsubstituted hydrocarbon radicals. Preference is given to pure hydrocarbon radicals, preferably having 1 to 16 carbon atoms. Selected examples of suitable hydrocarbon radicals R¹⁷ are alkyl radicals, such as the methyl, ethyl, n-propyl, isopropyl, n-butyl, isobutyl, tert-butyl, n-pentyl, isopentyl, neopentyl, tert-pentyl radical, hexyl radicals, such as the n-hexyl radical, heptyl radicals, such as the n-heptyl radical, octyl radicals, such as the n-octyl radical and isooctyl radicals, such as the 2,2,4-trimethylpentyl radical, nonyl radicals, such as the n-nonyl radical, decyl radicals, such as the n-decyl radical, dodecyl radicals, such as the n-dodecyl radical, and octadecyl radicals, such as the n-octadecyl radical, cycloalkyl radicals, such as cyclopentyl, cyclohexyl, cycloheptyl and methylcyclohexyl radicals, aryl radicals, such as the phenyl, naphthyl, anthryl and phenanthryl radical, alkaryl radicals, such as tolyl radicals, xylyl radicals and ethylphenyl radicals, and aralkyl radicals, such as the benzyl radical and the β-phenylethyl radical. Preferred hydrocarbon radicals as R¹⁷ are methyl, n-propyl, isopropyl, phenyl, n-octyl, or isooctyl radicals, with the methyl, n-propyl, phenyl, and isooctyl radical more preferred, and the methyl and phenyl radical particularly preferred.

### Component (B)

The condensation-crosslinkable silicone composition X1 preferably contains 15-45% by weight of one or more silanes B, preferably 20% to 40% by weight, in each case based on the total amount of components (A) and (B).

Preferably, at least 50% by weight, preferably at least 70% by weight, more preferably at least 90% by weight, particularly preferably at least 95% by weight, of silanes B, based on the total mass of all silanes B, satisfy the characteristic a=3 or 4; in a preferred embodiment at least 30% by weight, preferably at least 40% by weight, more preferably at least 50% by weight, of at least one silane B where a=4 is present, in each case based on the total mass of all silanes B.

Examples of suitable radicals R are hydrocarbon radicals having 1 to 16 carbon atoms, which can also be substituted. Particularly suitable and thus preferred are methyl, ethyl, isopropyl, and tert-butyl radicals and the p-nitrophenyl radical, with particular preference given to the methyl and ethyl radical.

The radicals R¹ may independently of one another be substituted or unsubstituted, monovalent hydrocarbon radicals. Preference is given to pure hydrocarbon radicals, preferably having 1 to 16 carbon atoms. Selected examples of suitable hydrocarbon radicals R¹ are alkyl radicals, such as the methyl, ethyl, n-propyl, isopropyl, n-butyl, isobutyl, tert-butyl, n-pentyl, isopentyl, neopentyl, tert-pentyl radical, hexyl radicals, such as the n-hexyl radical, heptyl radicals, such as the n-heptyl radical, octyl radicals, such as the n-octyl radical and isooctyl radicals, such as the 2,2,4-trimethylpentyl radical, nonyl radicals, such as the n-nonyl radical, decyl radicals, such as the n-decyl radical, dodecyl radicals, such as the n-dodecyl radical, and octadecyl radicals, such as the n-octadecyl radical, cycloalkyl radicals, such as cyclopentyl, cyclohexyl, cycloheptyl and methylcyclohexyl radicals, aryl radicals, such as the phenyl, naphthyl, anthryl and phenanthryl radical, alkaryl radicals, such as tolyl radicals, xylyl radicals and ethylphenyl radicals, and aralkyl radicals, such as the benzyl radical and the β-phenylethyl radical. Preferred hydrocarbon radicals as R¹ are methyl, n-propyl, isopropyl, phenyl, n-octyl, or isooctyl radicals, with the methyl, n-propyl, phenyl, and isooctyl radical more preferred, and the methyl and phenyl radical particularly preferred.

The condensation-crosslinkable silicone composition X1 may contain further solid or liquid constituents I, with the proviso that the condensation-crosslinking of the silicone compositions X1 and the formation of the shell H are unimpaired.

Examples of further constituents I include catalysts, active and inactive fillers, inhibitors, heat stabilizers, solvents, plasticizers, color pigments, soluble dyes, sensitizers, photoinitiators, adhesion promoters, conductivity additives, cosmetic substances, fragrances, medicinal or cosmetic active substances, fluorescent dyes, fungicides, fragrances, rheological additives, corrosion inhibitors, oxidation inhibitors, light stabilizers, heat stabilizers, flame-retardant agents, agents for influencing electrical properties, and agents for improving thermal conductivity.

These constituents can remain in the core of the hollow particle P and thereby be encapsulated, stored, transported or selectively released.

### Particulate Solid F

The particulate solid F used according to the invention is preferably in the form of particles that are solid at 20° C. and at the pressure of the ambient atmosphere, i.e. 1013 hPa.

The particulate solid F preferably has a solubility in water at pH 7.33, an electrolyte background of 0.11 mol, and a temperature of 37° C. of less than 0.1 g/l, more preferably of less than 0.05 g/l, at the pressure of the ambient atmosphere, i.e. 1013 hPa.

The particulate solid F preferably has a molar mass of greater than 10 000 g/mol, more preferably a molar mass of from 50 000 to 50 000 000 g/mol, in particular from 100 000 to 10 000 000 g/mol, in each case measured preferably by static light scattering.

The particulate solid F preferably has a BET specific surface area of 30 to 500 m²/g, more preferably 100 to 300 m²/g. The BET surface area is measured according to known methods, preferably in accordance with German industry standards DIN 66131 and DIN 66132.

The particulate solid F preferably has a Mohs hardness of greater than 1, more preferably of greater than 4.

The employed particulate solid F is preferably a metal oxide having a covalent bonding component in the metal-oxygen bond, for example solid oxides of the main group and transition group elements, such as one of main group 3, such as boron oxide, aluminum oxide, gallium oxide or indium oxide, or one of main group 4, such as silicon dioxide, germanium dioxide, tin oxide or dioxide, or lead oxide or dioxide, or an oxide of the transition group elements, such as titanium dioxide, zirconium dioxide, hafnium dioxide, cerium oxide or iron oxide.

The metal oxides employed according to the invention are preferably aluminum (III), titanium (IV) or silicon (IV) oxides, such as wet-chemically produced, for example precipitated, silicas or silica gels, or aluminum oxides, titanium dioxides or silicon dioxides produced in processes at elevated temperature, for example fumed aluminum oxides, titanium dioxides or silicas.

The median particle size of the particulate solid F or particle aggregates, if present, is here preferably less than the median diameter d₅₀ of the emulsion droplets formed according to the process of the invention in the absence of the finely particulate particles.

The median particle size of the particulate solid F is less than 1000 nm, preferably between 10 nm and 800 nm, more preferably between 50 nm and 500 nm, and most preferably between 75 nm and 300 nm, in each case measured as the median hydrodynamic equivalent diameter by photon correlation spectroscopy at 173° (backscattering) with a Nanosizer ZS from Malvern.

The methanol value of the particulate solid F is preferably less than 70, preferably less than 50, more preferably less than 40, and particularly preferably less than 30.

To determine the methanol value, defined mixtures of water with methanol are prepared and then the surface tensions of these mixtures determined using known methods. In a separate experiment, these water-methanol mixtures are overlayered with defined amounts of particles and shaken under defined conditions (for example, gentle manual shaking or shaking with a tumble mixer for approx. 1 minute). The water-alcohol mixture in which the particles do not yet sink and the water-alcohol mixture with a higher alcohol content in which the particles just sink are determined. The surface tension of the latter alcohol-water mixture gives the critical surface energy γ_{crit} as a measure of the surface energy γ of the particles. The methanol content in water gives the methanol value.

The carbon content of the particulate solid F is greater than 0% by weight, preferably 0.1-4% by weight, more preferably 0.25-3.5% by weight, and most preferably 0.5-3% by weight, measured by elemental analysis on the dry particulate solids.

In a preferred embodiment, the particulate solid F is a silica S.

Silicas S are preferably partially water-wettable fumed and precipitated silicas or mixtures thereof having a specific BET surface area of 30 to 500 m²/g, more preferably 100 to 300 m²/g, with fumed silica particularly preferred. The BET surface area is measured according to known methods, preferably in accordance with German industry standards DIN 66131 and DIN 66132.

Preferably, the silica S is surface-treated with a suitable hydrophobizing agent and is as a result hydrophobic. The hydrophobization must be carried out such that the silica S is still partially water-wettable. In accordance with the invention, this means that the methanol value of the silica S is less than 70, preferably less than 50, more preferably less than 40, and particularly preferably less than 30. As a result of surface treatment, preferred silicas S have a carbon content of at least 0.2% to max. 1.5% by weight, preferably between 0.4% and 1.4% by weight, more preferably between 0.6% and 1.3% by weight. The hydrophobic groups are for example Si-bonded methyl or vinyl groups. Methods for the hydrophobization of silicas are known to those skilled in the art.

Preference as silica S is given to silanized fumed silicas having a methanol value of less than 70, preferably less than 50, more preferably less than 40, and particularly preferably less than 30.

Very particular preference is given to partially water-wettable silicas as described in EP 1433749 A1 and DE 10349082 A1.

### Hollow Core K

The isolated and dried hollow particles P maintain a hollow core K. The average ratio of the average diameter of the hollow core K to the average diameter of the hollow particle P is here preferably greater than 0.2, preferably greater than 0.3, in each case determined as an average value of at least 5 individual particles by means of electron microscope images, for example TEM or SEM micrographs. The hollow core K can consist of a single cavity or a plurality of separate cavities.

The hollow core K is suitable for holding further constituents I.

### Shell H

The shell H comprises a silicone resin composition Z formed from the condensation-crosslinked silicone composition X and the particulate solid F. The shell H is formed through condensation-crosslinking of the emulsified condensation-crosslinkable silicone composition X1 at the boundary phase to the water-containing continuous phase of the emulsion. The boundary phase is stabilized by the particulate solid F, which is physically and/or chemically integrated into the developing shell H during condensation-crosslinking.

The shell H preferably has an average diameter of at least 50 nm, preferably at least 70 nm, in each case determined as an average value of at least 5 individual particles by means of electron microscope images, for example TEM or SEM micrographs.

### Catalyst K

In the case of less reactive condensation-crosslinkable silicone compositions X1, catalysts K are required in order to bring about the hydrolysis and condensation of the silicone resins A and silanes B, if necessary. Such catalysts are known to those skilled in the art. They can be either acids or bases or else metal catalysts, such as group IV transition metal catalysts, tin catalysts, such as those commonly used for accelerating hydrolyses, condensation reactions or transesterification reactions. As acids or bases, in addition to the known mineral acids and metal salts it is also possible to consider acidic or basic silanes or siloxanes.

Preferred basic catalysts are NaOH, KOH, ammonia and NEt₃. When using basic catalysts K, the pH of the reaction mixture is preferably in the range from pH 8 to pH 12.

Preferred acidic catalysts K are p-toluenesulfonic acid, aqueous or gaseous HCl, and sulfuric acid. When using acidic catalysts, the pH of the reaction mixture is preferably in the range from pH 1 to pH 5.

### Hollow Particles P

Hollow silicone resin particles of the prior art have a silicone resin shell that is obtained by coating and subsequent removal of a liquid or solid template. The silicone resin shell is hydrophobic and unsuitable for use in hydrophilic formulations and products, in particular unsuitable for use as an additive in aqueous formulations. The hollow particles P of the invention have an amphiphilic shell H of the silicone resin composition Z formed from the condensation-crosslinked silicone composition X and partially water-wettable solid F. The hollow particles P of the invention preferably have a methanol value of less than 80, preferably less than 60, more preferably less than 50, and particularly preferably less than 40. The hollow particles P of the invention can accordingly be easily processed in both hydrophilic and hydrophobic formulations and products.

The hollow particles P of the invention preferably have a BET greater than 4 m²/g, preferably greater than 10 m²/g, preferably greater than 20 m²/g.

The hollow particles P of the invention preferably have a bulk density of less than 0.28 g/cm³, preferably less than 0.25 g/cm³.

### Process for Producing the Hollow Particles P

The continuous phase preferably contains at least 80% by weight, in particular at least 90% by weight, of water.

Preferably, a three-phase mixture is formed in which an emulsion of sparingly water-soluble and water-immiscible condensation-crosslinkable silicone composition X1 is produced, which is stabilized in the water phase by partially hydrophobized silicas (Pickering emulsions). After emulsification, the condensation-crosslinkable silicone composition X1 undergoes crosslinking in a process suitable for production of the particles P. It may be necessary for the condensation-crosslinkable silicone composition X1 to be hydrolyzed, for example if it includes alkoxy- or acetoxy-substituted silanes or siloxanes. If the silicone compositions X1 are sufficiently reactive, the water already present may cause hydrolysis and subsequent condensation. The process must be carried out in such a way that no significant crosslinking takes place during emulsification, since otherwise no finely-divided emulsion is formed. In the case of less reactive condensation-crosslinkable silicone compositions X1, catalysts K are required in order to bring about the hydrolysis and condensation of the siloxanes and silanes, if necessary.

The second step of the process must be carried out such that the condensation-crosslinkable silicone composition X1 forming the discontinuous phase reacts with condensation-crosslinking at the interface to the continuous water-containing phase, forming the shell H of the hollow particles P, this being accompanied by physical and/or chemical bonding to the particulate solid F that stabilizes the boundary phase.

Those skilled in the art are aware that, before being dried for the first time, the newly formed hollow particles P in the dispersion are filled with liquid cleavage product of the condensation-crosslinking and/or with the continuous water-containing phase.

The size of the hollow particles P can be determined for example by the emulsifying technique, thus for example by variables such as the input shear energy, the volume fraction of the silicone composition X1, the amount of particulate solid F, the pH of the continuous water phase and ionic strength thereof, the viscosity, the dosing sequence, the dosing rate, or by the reaction regime, i.e. for example by the reaction temperature, the reaction time, and the concentrations of the employed raw materials.

The choice and amount of any hydrolysis/condensation catalyst used also has an influence on the particle size.

If further optional solid or liquid constituents I are present, these are in a first step preferably mixed homogeneously with the condensation-crosslinkable silicone composition X1 to form mixture B, before being emulsified with dispersion V in a further step and subsequently undergoing crosslinking to form the hollow particle P. This ensures that all optional solid or liquid constituents I are present in the interior of the droplets after emulsification and in the interior of the hollow particle P after crosslinking.

The Pickering emulsions E of mixture B are preferably essentially free of conventional organic surface-active substances that are non-particulate liquids and solids at room temperature and the pressure of the ambient atmosphere, such as nonionic, cationic, and anionic emulsifiers (“organic emulsifiers”).

What is meant here by organic emulsifiers is not particles and colloids, but rather molecules and polymers according to the definition of molecules, polymers, colloids, and particles given in “Dispersionen und Emulsionen” [Dispersions and emulsions], G. Lagaly, O. Schulz, R. Zindel, Steinkopff, Darmstadt 1997, ISBN 3-7985-1087-3, pp. 1-4.

In general, these organic emulsifiers have a size of less than 1 nm, a molar mass of <10 000 g/mol, a carbon content of >50% by weight, determinable by elemental analysis, and a Mohs hardness of less than 1.

At the same time, the organic emulsifiers, which are essentially absent in the emulsions of the invention, generally have a solubility in water in homogeneous or micelle form of greater than 1% by weight at 20° C. and the pressure of the ambient atmosphere, i.e. 900 to 1100 hP a.

The Pickering emulsions E of mixture B may contain such organic emulsifiers up to a maximum concentration of less than 0.1 times, preferably less than 0.01 times, more preferably less than 0.001 times, in particular less than 0.0001 times, the critical micelle concentration of these organic emulsifiers in the water phase; this corresponds to a concentration of these organic emulsifiers, based on the total weight of the dispersion of the invention, of less than 10% by weight, preferably less than 2% by weight, more preferably less than 1% by weight, in particular 0% by weight.

For the production of the particle-stabilized Pickering emulsion E in the first step, it is possible to use any method for producing emulsions known to those skilled in the art. However, it was found that emulsions particularly suitable for producing the hollow particles P can be obtained according to the following processes:

### Process 1:

A highly concentrated dispersion V is initially charged, the volume initially charged being such that it contains the total amount of solid F needed and only part of the volume of water.
The total volume of mixture B is metered in slowly under constant homogenization, for example by means of a high-speed stirrer, high-speed dissolver or a rotor-stator system.
The rest of the desired volume of water is then metered in slowly, optionally under constant homogenization, for example by means of a high-speed stirrer, high-speed dissolver or a rotor-stator system.

### Process 2:

The dispersion V is initially charged, the volume initially charged being such that it contains the total amount of solid F needed and water.
The total volume of mixture B is metered in slowly under constant homogenization, for example by means of a high-speed stirrer, high-speed dissolver, rotor-stator system or capillary emulsifier.

### Process 3:

The total volume of mixture B is initially charged.
A highly concentrated dispersion V is metered in slowly under constant homogenization, for example by means of a high-speed stirrer, high-speed dissolver or a rotor-stator system, the volume metered in being such that it contains the total amount of solid F needed and only part of the volume of water.
The rest of the desired volume of water is then metered in slowly, optionally under constant homogenization, for example by means of a high-speed stirrer, high-speed dissolver or a rotor-stator system.

### Process 4:

The total volume of mixture B is initially charged.
The dispersion V is metered in slowly under constant homogenization, for example by means of a high-speed stirrer, high-speed dissolver or a rotor-stator system, the volume metered in being such that it contains the total amount of solid F needed and water.

### Process 5:

The total volume of mixture B and of the dispersion V are initially charged, the volume initially charged being such that it contains the total amount of solid F needed and water.
Homogenization of both together, for example by means of a high-speed stirrer, high-speed dissolver or a rotor-stator system.

### Process 6:

The total volume of mixture B and of a highly concentrated dispersion V are initially charged, the volume initially charged being such that it contains the total amount of solid F needed and part of the volume of water.
Homogenization of both together, for example by means of a high-speed stirrer, high-speed dissolver or a rotor-stator system.
The rest of the desired volume of water is then metered in slowly, optionally under constant homogenization, for example by means of a high-speed stirrer, high-speed dissolver or a rotor-stator system.

Preference is given to processes 1, 2, 5, and 6, processes 2 and 5 being particularly preferred.

The homogenization is carried out preferably in at least one process step for at least 30 seconds, preferably at least 1 minute.

The dispersion V of the particulate solid F in water that forms the homogeneous phase in the emulsion of the invention can in principle be produced according to the known processes for producing particle dispersions, such as incorporation using agitators producing high shear, such as high-speed stirrers, high-speed dissolvers, rotor-stator systems, ultrasonic dispersers or ball/bead mills.

The concentration of the particulate solid F in the dispersion V is here between 1% and 80% by weight, preferably between 10% and 60% by weight, more preferably between 10% and 40% by weight, and most preferably between 12% and 30% by weight.

In an optional process step, the Pickering emulsion E is diluted with water, optionally under constant homogenization, for example by means of a high-speed stirrer, high-speed dissolver or a rotor-stator system.

The described processes can be carried out either continuously or discontinuously.

The temperature in the first step of emulsification is between 0° C. and 80° C., preferably between 10° C. and 50° C.

The emulsification process can be carried out at standard pressure, i.e. at 900 to 1100 hPa, at elevated pressure, or under reduced pressure. The process is preferably carried out at normal pressure.

The concentration of the particulate solid F in the three-phase mixture of dispersion V and mixture B from the first step is here between 1% and 80% by weight, preferably between 2% and 50% by weight, more preferably between 3% and 30% by weight, and most preferably between 4% and 20% by weight.

The concentration of the condensation-crosslinkable silicone composition X1 in the three-phase mixture of dispersion V and mixture B from the first step is here between 1% and 80% by weight, preferably between 20% and 76% by weight, more preferably between 40% and 72% by weight, and most preferably between 50% and 70% by weight.

The concentration of water in the three-phase mixture of dispersion V and mixture B from the first step is here between 5% and 80% by weight, preferably between 10% and 70% by weight, more preferably between 15% and 60% by weight, and most preferably between 20% and 40% by weight.

Starting from the three-phase mixture described above, the hollow particles P can be obtained in a second step according to the following process:

The three-phase mixture is preferably diluted by adding water to a water mass fraction of from 50% by weight to 90% by weight, preferably from 60% by weight to 80% by weight.

In the second step, the three-phase mixture is preferably stirred under low shear, for example by means of a slow-running dissolver, rotor-stator or paddle stirrer or shaken by means of suitable units until internal crosslinking of the hollow particles P is complete.

The duration of the second process step is preferably shorter than 120 h, preferably between 0 h and 48 h, more preferably 0.1 h to 24 h, and in a specific execution 0.25 h to 12 h.

The catalysts K that accelerate and complete crosslinking can optionally be added to the three-phase mixture of dispersion V and mixture B from the first step as mentioned above. These can be added directly to the discontinuous phase or continuous phase before production of the three-phase mixture, added during the emulsification, or they can be added later to the prepared three-phase mixture.

The amount of optionally added catalysts employed is within the typical range for the amount of catalyst.

The reaction temperature in the second step is between 0° C. and 100° C., preferably between 10° C. and 90° C., and more preferably between 20° C. and 80° C.

The reaction can optionally be carried out under an atmosphere of an inert gas such as nitrogen, argon or carbon dioxide. The oxygen content is in that case less than 15% by volume, preferably less than 10% by volume, and more preferably less than 5% by volume.

Water-soluble organic solvents, for example alcohols such as methanol, ethanol or isopropanol, or ketones such as acetone or MEK, or ethers such as THF or others may optionally be added to the three-phase mixture. These may be added in the first step or before or during the second step.

Dispersing aids, protective colloids and/or surfactants may optionally be added to the three-phase mixture. These may be added in the first step or before or during the second step.

The three-phase mixture preferably contains less than 5% by weight, more preferably less than 1% by weight, in particular less than 0.1% by weight, of dispersants, protective colloids, and surfactants. In a specific execution, the three-phase mixture is free of dispersing aids, protective colloids, and surfactants.

The three-phase mixture optionally comprises inorganic or organic electrolytes. These may be added either after the first step, during the second step, or after the end of the second step.

The ionic strength of the three-phase mixture is in this case between 0.01 mmol/l and 1 mol/l, preferably between 0.1 mmol/l and 500 mmol/l, and more preferably between 0.5 mmol/l and 100 mmol/l.

The surface of the hollow particles P may optionally be modified by treatment with reactive silanes or siloxanes. These may be added either immediately after the end of production of the Pickering emulsion in the first step, during the reaction phase or after the end of the reaction phase in the second step, before the isolation of the hollow particles P or after the isolation of the particles in the liquid or solid phase. The treatment must be carried out such that covalent chemical bonding of the silane or siloxane to the particles occurs. Suitable methods and processes are known to those skilled in the art.

The solids content of the hollow particles P in the three-phase mixture consisting of the sum total of the solids used and the polymerization product of the polyaddition-capable, polycondensable or polymerizable material is between 5% by weight and 70% by weight, preferably between 10% by weight and 50% by weight, and more preferably between 20% by weight and 40% by weight.

The three-phase mixture after the second step may optionally be stored for a continued period with stirring. This may be done for example by means of a paddle stirrer or anchor stirrer.

In a preferred embodiment, the hollow particles P are isolated, preferably by sedimentation, filtration or centrifugation, more preferably by filtration or centrifugation, particularly preferably by centrifugation.

After isolation, the hollow particles P are preferably washed with a wash liquid preferably selected from demineralized water, methanol, ethanol, and mixtures thereof.

In a preferred embodiment, the hollow particles P are isolated from the aqueous phase in powder form. This may be done for example by filtration, sedimentation, centrifugation or by removal of volatiles by drying in ovens or dryers or by spray drying or by applying an appropriate reduced pressure.

Spray drying allows very high fineness to be achieved in the particles P without further processing. Statically dried hollow particles P tend to form loose agglomerates, which can be deagglomerated by suitable milling processes, for example a ball mill or air-jet mill.

The aqueous dispersion of the hardened hollow particles can be used for all purposes for which aqueous dispersions are also used to date. The aqueous dispersion can be used for cosmetic and pharmaceutical applications, cleaning and cleansing compositions or applications involving altering the interface properties of solid and liquid substrates, for example hydrophobizing agents, adhesion promoters, release agents, paper coatings or foam-control agents, for the production of w/o/w or o/w/o multiple emulsions, for example as controlled-release systems or for the segregation of reactive substances.

The hardened hollow particles P are used particularly in cosmetic and medicinal products and as a lightweight filler in the plastics and ceramics sector.

The hollow particles P show very advantageous behavior, especially for cosmetic applications. They are not prone to agglomeration or blocking and are therefore extremely easy to spread and make the skin feel velvety smooth.

By comparison with noninventive filled particles that do not have a hollow core, the hollow particles P have a lower density or can be filled in the core with other functional substances, for example with active constituents, such as fragrances, care substances, vitamins, UV absorbers or active substances, and are able to transport and release said substances in a controlled manner.

By comparison with noninventive hollow particles that do not have an amphiphilic shell H formed from the silicone resin composition Z, silica-coated hollow particles can absorb a larger amount of functional substances on the silica surface, for example fragrances, care substances, vitamins or UV absorbers, or medicinal active substances, and are able to transport and release said substances in a controlled manner.

By comparison with noninventive particles that do not have an amphiphilic shell H formed from the silicone resin composition Z, the silica-coated particles show amphiphilic behavior, i.e. they are readily dispersible in both oily and aqueous liquids.

By comparison with noninventive particles that do not have an amphiphilic shell H formed from the silicone resin composition Z, the surface of the hollow particles of the invention is more readily wettable by liquids. As a result, they are able to be dispersed much more easily and quickly in liquids, for example in cosmetic formulations, and they also absorb liquids on the surface significantly more quickly and easily, for example they absorb sebum when applied cosmetically to the skin.

### Measurement Methods

Molecular weight distributions:

Molecular weight distributions are determined as the weight-average Mw and as the number-average Mn using the method of gel-permeation chromatography (GPC or size-exclusion chromatography (SEC)) with a polystyrene standard and a refractive index detector (RI detector). Unless otherwise stated, THF is used as eluent and DIN 55672-1 is followed. The polydispersity is the ratio Mw/Mn.
Solids content: 10 g of aqueous dispersion is mixed in a porcelain dish with the same amount of ethanol and evaporated to constant weight in an N₂-flushed drying oven at 150° C. The mass ms of the dry residue gives the solids content according to the expression: solids content/%=ms*100/10 g.
Median particle diameter (d50 value) and particle diameter:

The d₅₀ value was determined using a Camsizer X2 from Retsch Technology (measurement principle: dynamic image analysis according to ISO 13322-2, measurement range: 0.8 μm to 8 mm, analysis type: dry measurement of powders and granulates, dispersion pressure=2 bar).
Carbon content % C determined by elemental analysis for carbon; combustion of the sample at over 1000° C. in a stream of 02, detection and quantification of the resulting CO₂ in a Leco 244 IR analyzer.
Methanol value: For determination of the methanol value, defined mixtures of water and methanol are prepared. In a separate experiment, these water-methanol mixtures are overlayered with the same volume of dried particles and shaken under defined conditions (for example, gentle shaking by hand or with a tumble mixer for approx. 1 minute). The water-alcohol mixture in which the particles do not yet quite sink and the water-alcohol mixture with a higher alcohol content in which the particles just sink are determined. The latter methanol content in water gives the methanol value.
The kinematic viscosity is measured according to DIN 53019 at 25° C.

In the examples that follow, unless otherwise stated in each case, all amounts and percentages are based on weight, all pressures are 0.10 MPa (abs.), and all temperatures are 20° C.

### EXEMPLARY EMBODIMENTS

### Example 1: Production of an Aqueous Silica Dispersion

1300 g of partially hydrophobic fumed silica having a residual silanol content of 71% and a carbon content of 0.95% by weight, obtained by reacting a hydrophilic starting silica having a BET specific surface area of 200 m²/g (available under the HDK® N20 name from Wacker-Chemie GmbH, Munich) with dimethyldichlorosilane according to EP 1433749 A1, is stirred a little at a time into 5200 g of demineralized water in a dissolver at 650 rpm. At the end of the addition of the silica, the mixture is further dispersed at 650 rpm for a further 60 min. A highly viscous dispersion having a solids content of 20% and a pH of 4.2 is obtained.

### Example 2: General Procedure for Producing a Pickering Emulsion of the Condensation-Crosslinkable Silicone Compositions X1 Using an Ultra-Turrax®

Step 1: The silica dispersion described in example 1 is weighed out in a suitable 1000 ml stainless steel vessel and stirred with an Ultra-Turrax® T50 at 10 000 rpm for 10 min. The viscosity of the dispersion decreases during this operation. Demineralized water is optionally added and mixed in homogeneously. The components of the condensation-crosslinkable silicone compositions X1 according to examples 4 to 7 are mixed with a laboratory stirrer and added to the stirred silica dispersion and then homogenized using the Ultra-Turrax for a total of 10 min at 10 000 rpm with ice cooling. The temperature of the mixture should not rise above 35° C. during this operation. If the temperature exceeds 35° C., mixing is halted for cooling. Care must also be taken to ensure that the emulsion that forms remains flowable. If necessary, a small amount (approx. 50 ml) of water for dilution is added, multiple times if needs be. A white, highly viscous mass (emulsion (E)) results.

Step 2: The highly viscous mass from step 1 is diluted to a silicone oil content of 30% by adding three equal-sized portions of demineralized water. After each portion of demineralized water the mixture is stirred at 6000 rpm for 3 minutes. A freely mobile, white O/W emulsion forms.

### Example 3: General Procedure for Producing the Hollow Particles from Inventive Examples 4 to 7 and the Noninventive Comparative Examples V1 to V3

1.5 g of p-toluenesulfonic acid is added to 250 g of the polycondensation-capable Pickering emulsion (E) produced according to the general procedure from example 2. The reaction mixture is stirred at room temperature for 24 h. A white, freely mobile dispersion results. The particles are filtered off and dried in a drying oven at 60° C. for 24 h. A fine white powder is obtained.

Silicone resin S1: Methoxy-group-containing oligomeric condensation product of methyltrimethoxysilane having a methoxy group content of approx. 30% by weight and the composition [MeSiO_{3/2}]₂₆ [MeO_{1/2}]₂₃ (molecular weight according to SEC (eluent: toluene): Mw=2300 g/mol; Mn=600 g/mol; viscosity (kinematic, DIN 51562, 25° C.) 25 mm²/s)

Silicone resin S2: Ethoxy-group-containing oligomeric condensation product of methyltriethoxysilane having an ethoxy group content of approx. 36% by weight and the composition [MeSiO_{3/2}]₂₃ [MeO_{1/2}]₂₇ (molecular weight according to SEC (eluent: toluene): Mw=2560 g/mol; Mn=900 g/mol; viscosity (kinematic, 25° C.) 22 mm²/s.

Silicone resin S3: Methoxy-group-containing oligomeric condensation product of phenyltrimethoxysilane and dimethyldimethoxysilane having an average molecular weight Mw of 1030 g/mol (number-average Mn=730; polydispersity 1.4) and a viscosity of 140 mm²/s (25° C.), bearing on the surface 12.3% by weight of Si-bonded methoxy groups and 0.24% by weight of Si-bonded OH groups and consisting on average of 59 mol % of PhSiO_{3/2} units and 41 mol % of Me₂SiO_{2/2} units, the methoxy groups being distributed among the stated structural units.

The properties and results for the examples are summarized in Table 1. The constituents of the silicone resin components (A) and silane components (B) are given in parts by weight:

TABLE 1

| Example | | 4 | 5 | 6 | 7 | V1 | V2 | V3 |
|---|---|---|---|---|---|---|---|---|
| Silicone resin | Silicone | — | — | — | 2 | 1 | — | — |
| (A) | resin S1 | | | | | | | |
| | Silicone | 2 | 2 | — | — | — | 1 | — |
| | resin S2 | | | | | | | |
| | Silicone | — | — | 2 | — | — | — | — |
| | resin S3 | | | | | | | |
| Silane (B) | Tetra- | 1 | 0.5 | 1 | 1 | — | — | 1 |
| | ethoxysilane | | | | | | | |
| | Methyltri- | — | 0.5 | — | — | — | — | — |
| | ethoxysilane | | | | | | | |
| Inventive | | yes | yes | yes | yes | no | no | no |
| (yes/no) | | | | | | | | |
| Formation of | | yes | yes | yes | yes | yes | yes | no |
| spherical | | | | | | | | |
| microparticles | | | | | | | | |
| Form | | hollow | hollow | hollow | hollow | filled | filled | — |
| (filled/hollow) | | | | | | | | |
| Particle size | | 2.0 | 2.1 | 4.4 | 1.8 | 3.6 | 3.1 | — |
| d50 (mm) | | | | | | | | |
| Bulk density | | 0.23 | 0.21 | n.d. | n.d. | 0.3 | n.d. | — |
| in g/cm³ | | | | | | | | |
| BET in m²/g | | 186 | 185 | n.d. | n.d. | 10 | n.d. | — |

### USE EXAMPLES

### Example 8: Use in Coatings

A silicone coating according to the invention was produced. This was done by homogeneously mixing 2 parts of the inventive hollow particles from example 4 with 98 parts of Elastosil® RT 601 A/B (a pourable, addition-crosslinking two-component silicone rubber vulcanizable at room temperature, obtained from Wacker Chemie AG, Munich, Germany) by stirring at 6000 rpm for 10 min with a dissolver, maintaining the temperature at 20° C. The resulting mass was applied to a glass plate with a 10 μm doctor blade. A transparent smooth coating is obtained.

### Example 9: Optical Evaluation in Cosmetic Use

100 mg of the inventive hollow particles from example 5 was evenly distributed on a circular area 4 cm in diameter on the uncleaned forearm of a human test subject. A dry, even, optically homogeneous and slightly whitish skin surface is obtained. This is a sign that sebum present is completely adsorbed from the skin surface.

### Example 10: Use as a Lightweight Filler

5 parts of the inventive hollow particles from example 5 and 95 parts of Elastosil® LR 3003/40 A/B (a pasty addition-crosslinking two-component silicone rubber, obtained from Wacker Chemie AG, Munich, Germany) were homogeneously mixed with a laboratory stirrer. A test specimen 4 cm in diameter and 0.6 cm in height was then cured in a suitable mold at 165° C. for 30 min. The density of the test specimen is 1.01 g/ml, measured according to DIN EN ISO 1183-1 A.

### Comparative Example V4

A test specimen was produced in analogous manner to example 10, but without addition of an inventive hollow particle. The density of the reference test specimen is 1.09 g/ml, measured according to DIN EN ISO 1183-1 A.

1-16. (canceled)

17. A hollow particle P, comprising:
wherein the hollow particle P is constructed of a hollow core K and a shell H comprising a silicone resin composition Z that comprises a condensation-crosslinked silicone composition X and a particulate solid F.

18. The hollow particle P of claim 17, wherein the median particle diameter d50 of which is in the range 0.1-100 μm, measurable using a Camsizer X2 from Retsch Technology (measurement principle: dynamic image analysis according to ISO 13322-2, measurement range: 0.8 μm to 8 mm, analysis type: dry measurement of powders and granulates, dispersion pressure=2 bar).

19. The hollow particle P of claim 17, wherein the hollow particle P has a sphericity SPHT3 of at least 0.8, determinable in accordance with ISO 9276-6 using a Camsizer X2 from Retsch Technology.

20. The hollow particle P of claim 17, wherein the shell H has an average diameter of at least 50 nm, in each case determined as an average value of at least 5 individual particles by means of electron microscope images, for example TEM or SEM micrographs.

21. The hollow particle P of claim 17, wherein the average ratio of the average diameter of the hollow core K to the average diameter of the hollow particle P is greater than 0.2, determinable as an average value of at least 5 individual particles by means of electron microscopic images, for example TEM or SEM micrographs.

22. The hollow particle P of claim 17, wherein the particulate solid F is selected from aluminum (III), titanium (IV) and silicon (IV) oxides.

23. The hollow particle P of claim 17, wherein the particulate solid F is partially water-wettable fumed or precipitated silica or mixture thereof having a specific BET surface area of 30 to 500 m²/g, measurable in accordance with German industry standards DIN 66131 and DIN 66132.

24. The hollow particle P of claim 17, wherein the methanol value of the particulate solid F is less than 70, wherein defined mixtures of water with methanol are prepared for the determination of the methanol value and, in a separate experiment, these water-methanol mixtures are overlayered with the same volume of dried particles and shaken under defined conditions;
wherein the water-alcohol mixture in which the particles do not yet quite sink and the water-alcohol mixture with a higher alcohol content in which the particles just sink are determined; and
wherein the latter methanol content in water gives the methanol value.

25. A process for producing the hollow particles P, comprising:
wherein in a first step, a dispersion V comprising particulate solid F and water is mixed with condensation-crosslinkable silicone composition X1, which comprises alkoxy-group-containing silicone resin A that is liquid at 20° C. and alkoxy-group-containing silane B, to form a continuous water-containing phase and a discontinuous phase comprising condensation-crosslinkable silicone composition X1; and
wherein in a second step, the silicone composition X1 undergoes crosslinking in the discontinuous phase to form the silicone composition X, resulting in the formation of the hollow particles P.

26. The process of claim 25, wherein the condensation-crosslinkable silicone composition X1 comprises, respectively based on the total amount of components (A) and (B),
(A) 50-90% by weight of at least one silicone resin A, composed of units of formulas (Ia), (Ib), (VII), and (Id) 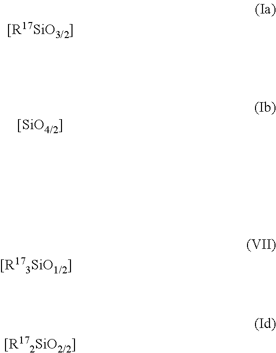
wherein R¹⁷ represent identical or independently different monovalent, substituted or unsubstituted organic radicals bearing or not bearing functional groups, or an —OH or a hydrogen radical;
wherein at least 20 mol % of the formula (Ia) or (Ib) or of a mixture of the two is present in (A);
wherein not more than 50 mol % of the formula (Ib) is present in (A);
wherein alkoxy groups are present as R¹⁷ in (A) to an extent of at least 5% by weight;
wherein (A) is liquid at 20° C.;
(B) 10-50% by weight of at least one silane B of the general formula 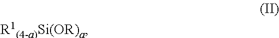
wherein R is a hydrocarbon radical having 1 to 16 carbon atoms, the carbon chain of which may be interrupted by non-adjacent —O— groups;
wherein R¹ represents monovalent hydrocarbon radicals that are identical or independently different from one another;
wherein a represents the values 2, 3 or 4; and
wherein at least 20% by weight of silanes B, based on the total mass of all silanes B, satisfy the characteristic a=3 or 4.

27. The process of claim 25, wherein the alkoxy group radicals R¹⁷ of the silicone resin A are selected from methoxy, ethoxy, isopropoxy, n-butoxy, and tert-butoxy radicals.

28. The process of claim 25, wherein the radicals R of the silane B are selected from methyl, ethyl, isopropyl, and tert-butyl radicals.

29. The process of claim 25, wherein the particulate solid F is selected from aluminum (III), titanium (IV) and silicon (IV) oxides.

30. The process of claim 25, wherein the median particle size of the particulate solid F is less than 1000 nm, measured as the median hydrodynamic equivalent diameter by photon correlation spectroscopy at 173° (backscattering) with a Nanosizer ZS from Malvern.

31. The process of claim 25, wherein the particulate solid F is partially water-wettable fumed or precipitated silica or mixture thereof having a specific BET surface area of 30 to 500 m²/g, measurable in accordance with German industry standards DIN 66131 and DIN 66132.

32. The process of claim 25, wherein the particulate solid F has a methanol value of less than 70, wherein defined mixtures of water with methanol are prepared for the determination of the methanol value and, in a separate experiment, these water-methanol mixtures are overlayered with the same volume of dried particles and shaken under defined conditions;
wherein the water-alcohol mixture in which the particles do not yet quite sink and the water-alcohol mixture with a higher alcohol content in which the particles just sink are determined; and
wherein the latter methanol content in water gives the methanol value.
